(19) United States
(12) Patent Application Publication
(10) Pub. No.: US 2023/0028699 A1
(43) Pub. Date: Jan. 26, 2023

(54) Urinary Output Measuring System

(71) Applicant: C. R. Bard, Inc., Franklin Lakes, NJ (US)

(72) Inventors: Jason Jishen Cheng, Avondale Estates, GA (US); Christopher K. Brooks, Lawrenceville, GA (US); Robert M. Hine, Covington, GA (US)

(21) Appl. No.: 17/863,223

(22) Filed: Jul. 12, 2022

Related U.S. Application Data
(60) Provisional application No. 63/221,804, filed on Jul. 14, 2021.

(51) Int. Cl. A61B 5/20; A61B 5/00

(52) U.S. Cl. CPC A61B 5/208; A61B 5/0022; A61B 5/6891; A61B 2562/0247

(57) ABSTRACT

A urinary output measuring system includes a system module, a fluid collection bag, and a pressure measurement tube having an open end disposed at the bottom interion of the fluid collection bag. The bag is configured to receive urine from a patient as delivered via a urinary catheter. The system module includes a pressure sensor, an air pump, and a valve. Processors perform operations of the system as defined by logic. A manifold defines fluid communication between the tube, the air pump, the pressure sensor, and the valve. Operations and methods include purging fluid from the tube, determining a pressure within the tube, and converting the pressure to a volume of urine within the bag. The bag includes a vent having includes a sterilizing filer and a hydrophobic membrane. The system module is configured to couple with a bed and suspend the bag.

### PRIORITY

This application claims the benefit of priority to U.S. Provisional Application No. 63/221,804, filed Jul. 14, 2021, which is incorporated by reference in its entirety into this application.

### BACKGROUND

Urinary output measuring systems measure urinary output in a variety of ways. Some systems measure urine by weight but require a significant footprint in a hospital environment. Other systems require modifications to the system including active pumping and multi-lumen tubing, complicating setup of the system. Some systems rely on clinicians to manually measure and record the urine volume. It would be beneficial to the patient and the clinician to have a urinary output measuring system that is compact, having automated urinary output measuring capabilities, and could be adapted to current urinary output measuring systems. Disclosed herein is a system and method of use that address the foregoing.

### SUMMARY

Disclosed herein is a urinary output measuring system, that according to some embodiments, includes a collection container configured to receive therein urine from a patient via a urinary catheter and a pressure measurement tube defining a first end and an opposite second end, where the second end is disposed within the collection container such that the second end is located at the bottom of the collection container. The system further includes a system module that includes (i) a pressure sensor in fluid communication with the pressure measurement tube so as to detect a pressure within the pressure measurement tube, (ii) an air pump fluidly coupled with the pressure measurement tube adjacent the first end, and (iii) a console operatively coupled with the pressure sensor and the air pump. The console includes one or more processors and a non-transitory computer-readable medium having stored thereon logic that, when executed by the one or more processors, causes operations of the system that include determining a volume of the urine collected within the collection container. Determining the volume of the urine includes (i) activating the air pump to cause a flow of air from the first end to the second end of the pressure measurement tube, thereby purging the urine from the pressure measurement tube; (ii) obtaining a pressure measurement of the air when the urine is purged from the pressure measurement tube, where the pressure measurement defines by a hydrostatic pressure of the urine collected within the collection container; and (iii) calculating a volume of the urine collected within the collection container based on the pressure measurement in combination with a geometry of the collection container. In some embodiments, the collection container is a flexible bag.

In some embodiments, the system further includes a display coupled with the console, and the operations further include indicating the volume of the urine on the display.

In some embodiments, the console further includes a wireless module, and the operations further include wirelessly communicating the volume of urine to at least one of a computing device or an electronic medical record system.

In some embodiments, the system module further includes a housing for containing the pressure sensor, the air pump, the console, and the display, and the housing is configured to attach to a bed.

In some embodiments, the collection container is configured to attach to the housing such that the collection container is suspended from the housing.

In some embodiments, the container includes a hook and the housing includes a hook pocket configured to receive the hook.

In some embodiments, the collection container defines a closed volume, and the pressure measurement tube extends through a wall of the collection container such that the pressure measurement tube forms a seal with the wall.

In some embodiments, the system further includes a drainage tube fluidly coupleable with the collection container, the drainage tube coupleable with the urinary catheter to enable flow of urine from the catheter to the collection container. In some embodiments, the drainage tube is fixedly attached to the collection container.

In some embodiments, the collection container includes a vent configured to enable passage of air out of the collection container, where the vent includes a vent filter extending across a flow path of the vent, where the vent filter defines a sterile barrier, and where the vent filter includes a hydrophobic membrane.

In some embodiments, the air pump includes a pump filter extending across a flow path of the air pump, the pump filter defining a sterile barrier.

In some embodiments, the system further includes valve fluidly coupled with the pressure measurement tube such that, when the valve is in an opened state, air is allowed to flow into and out of the pressure measurement tube. In such embodiments, the valve is operatively coupled with the console, and the logic is configured to transition the valve between the opened state and a closed state. The operations further include transitioning the valve from the opened state to the closed state before activating the air pump and transitioning the valve from the closed state to the open state after obtaining the pressure measurement.

In some embodiments, the valve includes a valve filter extending across a flow path of the valve, where the valve filter defines a sterile barrier.

In some embodiments, obtaining a pressure measurement of the air includes (i) obtaining a plurality of pressure measurements, (ii) calculating an average of the plurality of pressure measurements, and calculating a volume of the urine collected within the collection container includes calculating the volume of the urine based on an average of the plurality of pressure measurements.

In some embodiments, the system further includes a manifold having a frame structure and a number of fluid passageways that define the fluid communication between pressure measurement tube and each of the pressure sensor, the air pump, and the valve, where the pressure sensor, the air pump, and the valve are fixedly attached to the manifold, and the pressure measurement tube is detachably coupled with the manifold.

In some embodiments, the operations further include determining a volume of urine collected within the collection container multiple times according to a predefined determination schedule and/or in response to an input from the user.

Also disclosed herein is a method of determining a volume of urine collected within a bag. According to some embodiments, the method includes (i) purging the urine from a pressure measurement tube extending between a pressure sensor and a bottom interior of the bag, (ii) obtaining a pressure measurement from the pressure sensor, the pressure measurement related to a hydrostatic pressure of the urine, and (iii) calculating the volume of the urine based on the pressure measurement and a geometry of the bag.

In some embodiments of the method, purging the urine from the pressure measurement tube includes pumping sterile air into the tube so that air bubbles exit the pressure measurement tube at the bottom interior of the bag.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a perspective view of a urinary output measuring system, in accordance with some embodiments;

FIG. 2 illustrates a cross sectional view of the urinary output measuring system of FIG. 1, in accordance with some embodiments;

FIG. 3 illustrates a block diagram of some components of the urinary output measuring system of FIG. 1 including the console, in accordance with some embodiments;

FIG. 4A illustrates a cross sectional view of the urinary output measuring system of FIG. 1 depicting an exemplary method of measuring a volume of fluid within the fluid collection bag, in accordance with some embodiments;

FIGS. 4B-4C illustrate a cross sectional view of the urinary output measuring system of FIG. 1 depicting another exemplary method of measuring the volume of fluid within the fluid collection bag, in accordance with some embodiments;

FIG. 5 illustrates a plan view of the urinary output measuring system of FIG. 1 further including a display, in accordance with some embodiment; and

FIG. 6 illustrates a flow chart of an exemplary method of measuring the volume of fluid within the fluid collection bag, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

Unless defined otherwise, all technical and scientific terms used herein have the same meaning as commonly understood by those of ordinary skill in the art.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

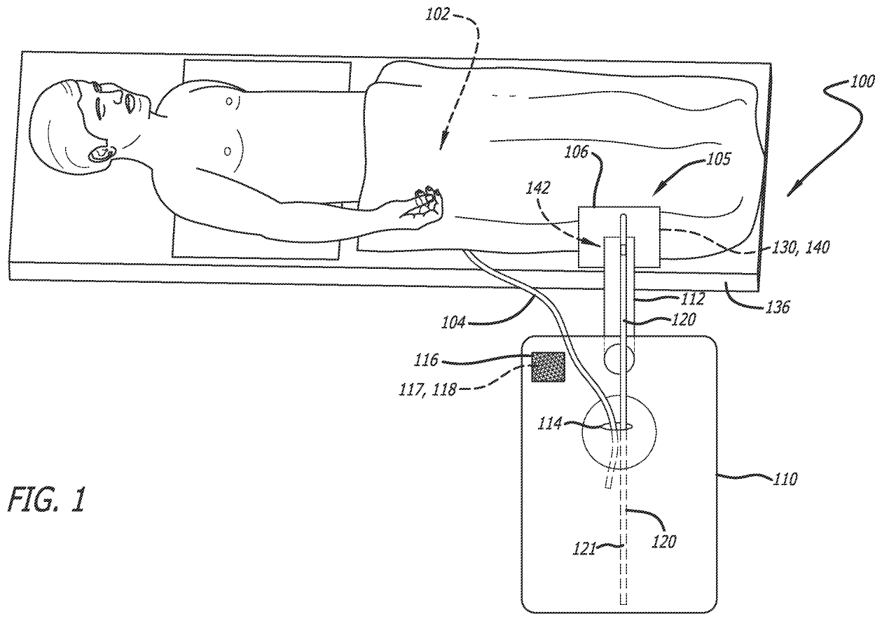

FIG. 1 illustrates a perspective view of a urinary output measuring system (system) 100, in accordance with some embodiments. The system 100 is generally configured to measure the volume of urine output from a patient. The system 100 generally includes a system module 105 and a fluid collection bag 110. The term fluid as used herein refers more specifically to urine from a patient, and as such, the terms “fluid” and “urine” are used interchangeably. The system 100 includes further a drainage tube 104 fluidly coupled with the fluid collection bag 110. The drainage tube 104 is coupleable to a urinary catheter 102 to provide a flow path for urine to flow from the patient to the fluid collection bag 110. In some embodiments, the system 100 may include the urinary catheter 102. In some embodiments, the fluid collection bag 110 may include or be coupled with a fluid collection bag holder 112 (e.g., a hook). The fluid collection bag 110 may be suspended from the fluid collection bag holder 112, allowing the volume of fluid to move from the drainage tube 104 into the fluid collection bag 110 under gravity flow. In some embodiments, the volume of fluid may move from the drainage tube 104 into the fluid collection bag 110 under negative pressure flow or the like. The fluid collection bag 110 may be a flexible bag or rigid container. The system 100 further includes a pressure measurement tube 120 inserted through a first opening 114 in the fluid collection bag 110. The first opening 114 may be configured to form a fluid tight seal with the pressure measurement tube 120 when the pressure measurement tube 120 is inserted therein. In some embodiments, the first opening 114 may be located on a side wall or at the top of the fluid collection bag 110. The pressure measurement tube 120 includes a fluid tube lumen 121 therein. The system 100 further includes a pressure sensor 130 configured to detect pressure within the fluid tube lumen 121. The pressure measurement tube 120 may be fluidly coupled with a manifold 140 where the manifold includes fluid passages to enable fluid communication between the pressure sensor 130 and the pressure measurement tube 120. In some embodiments, the manifold 140 may define a frame to which components such as the pressure sensor 130 may be attached. In some embodiments, the pressure sensor 130 may be fixedly attached to the manifold 140.

The system module 105 may include a housing for containing components of the system module 105 such as the pressure sensor 130 and the manifold 140. In some embodiments, the housing 106 may be configured to selectively couple to a hospital bed 136, hang on a wall, or sit on a specially designed stand close to the hospital bed 136. In some embodiments, the housing 106 may be coupled to the hospital bed 136 (e.g., a bed rail) via a press fit, a snap fit, an interference fit, a magnetic fit or the like. The housing 106 may include a holder pocket 142 (e.g., a hook receiver) configured to receive therein the fluid collection bag holder 112. The system 100 is configured to utilize the pressure measurement tube 120, the pressure sensor 130, and the manifold 140 to detect and measure the volume of fluid (i.e., urine) within the fluid collection bag 110, as will be described in more detail herein. One advantage of the system 100 as disclosed herein is that the system module 105 including the electronics may be spaced away from the fluid collection bag 110 and the patient. Furthermore, the only operational connection between the system module 105 and the fluid collection bag 110 is the pressure measurement tube 120.

In some embodiments, the fluid collection bag 110 may include a vent 116 configured to enable passage of air into and/or out of the fluid collection bag 110. The vent 116 includes a vent filter 117 extending across a flow path of the vent 116 to prevent contaminants from entering the fluid collection bag 110. As such, the vent filter 117 may define a sterile barrier of the fluid collection bag 110. In some embodiments, the vent filter 117 may define a pore size of 0.2 microns or less. The vent filter 117 may also include a hydrophobic membrane 118 configured to prevent urine from exiting the fluid collection bag 110 via the vent 116.

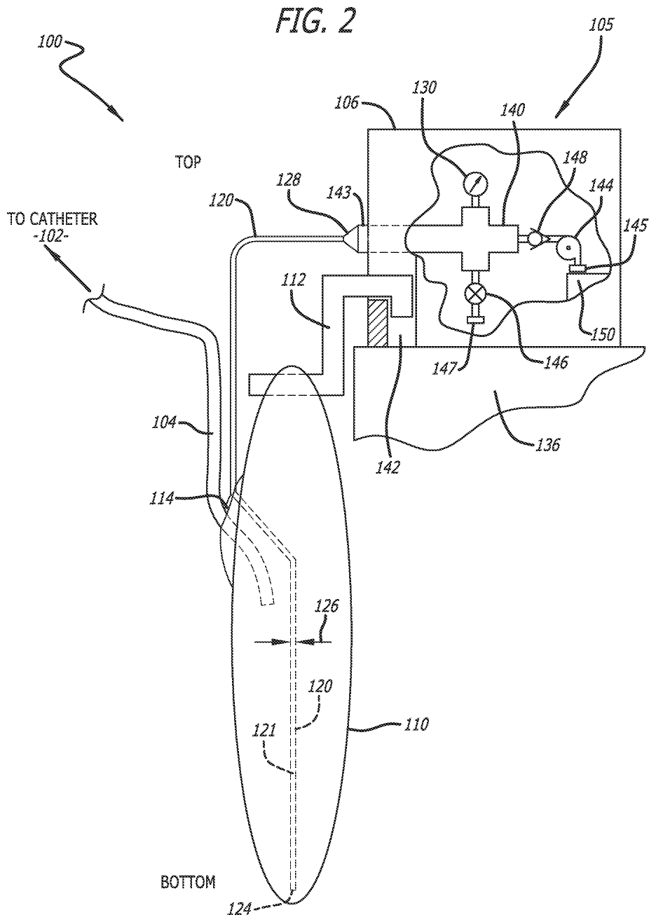

FIG. 2 illustrates a cross sectional view of the urinary output measuring system 100, according to some embodiments. The system 100 further includes an air pump 144 fluidly coupled with the pressure measurement tube 120 via the manifold 140. The air pump 144 is configured to pump air into the pressure measurement tube 120 to purge urine from the pressure measurement tube 120. The pressure measurement tube 120 is selectively coupled with an air outlet 143 of the manifold 140. In some embodiments, the pressure sensor 130 may also be coupled with the pressure measurement tube 120 via the manifold 140. In some embodiments, the air pump 144 may include a pump filter 145 extending across the flow path of the air pump 144, and the pump filter 145 may resemble the components and functionality of the vent filter 117. In some embodiments, the air pump 144 may be fixedly attached to the manifold 140.

In some embodiments, the system 100 may include electro-mechanical valve 146 operatively coupled with the pressure measurement tube 120. The valve 146 is configured to allow air to enter and/or exit the pressure measurement tube 120 through the valve 146 when the valve 146 is disposed in an opened state. Similarly, the valve 146 may prevent air from entering and/or exiting the pressure measurement tube 120 through the valve 146 when the valve 146 is disposed in a closed state. The valve 146 may include a valve filter 147 extending across the flow path of the valve 146, and the valve filter 147 may resemble the components and functionality of the vent filter 117. In some embodiments, the valve 146 may be coupled with an inlet port of the air pump 144. In some embodiments, the valve 146 may be fixedly attached to the manifold 140.

The system module 105 includes a console 150 in communication with each of the pressure sensor 130, the air pump 144, and the valve 146. The valve 146 may selectively allow and a prevent passage of air through the valve 146 based on an electrical signal. The console 150 may be configured to receive one or more detected pressure measurement values from the pressure sensor 130 and logic of the console 150 may be configured to correlate the detected measurement values with a volume of urine within the fluid collection bag 110, as will be described in more detail herein. The holder pocket 142 may be configured to slidably receive therein the fluid collection bag holder 112, as illustrated in FIG. 2.

In some embodiments, one or more of the pressure measurement tube 120, the fluid collection bag 110, and the fluid collection bag holder 112 may be disposable. The pressure measurement tube 120 may be detachably coupled to the manifold 140. In some embodiments, the pressure measurement tube 120 may be formed separately and coupled with the fluid collection bag 110 upon point of use. In other embodiments, the pressure measurement tube 120 may be formed integrally into the fluid collection bag 110 and coupled to the manifold 140 upon point of use. The pressure measurement tube 120 is inserted into the fluid collection bag 110 so that an open end of the pressure measurement tube 120 is disposed near a bottom of the fluid collection bag 110. The pressure measurement tube 120 may be constructed of any rigid or flexible material (e.g., PVC or the like) that will not kink, occlude, or permanently deform during use. The pressure measurement tube 120 may include a fluid tube diameter 126 large enough so that the level of fluid within the fluid collection bag 110 and the level of fluid within the pressure measurement tube 120 are substantially equal when the pressure measurement tube 120 is deployed within the fluid collection bag 110 and valve 146 is opened. The pressure measurement tube 120 may include a fluid tube connector 128 configured to be detachably coupled to the air outlet 143, providing a fluid tight seal between the pressure measurement tube 120 and the air outlet 143. The fluid tube connector 128 may be coupled to the manifold 140 via a press fit, a snap fit, an interference fit, a twist fit, a screw fit, or the like.

In some embodiments, the pressure sensor 130 may be coupled to the outside of the pressure measurement tube 120 and extend into the fluid tube lumen 121 to detect pressure within the fluid tube lumen 121 or may exist within the fluid tube lumen 121 without significantly impeding fluid flow or air flow therethrough. The pressure sensor 130 may be coupled with the pressure measurement tube 120 at any location along the pressure measurement tube 120 or via the manifold 140. In some embodiments, the system 100 may include more than one pressure sensor 130 coupled with the pressure measurement tube 120.

In some embodiments, the air pump 144 may include a check valve 148 configured to allow air to flow toward the manifold 140 and prevent air from flowing away the manifold 140, i.e., out of the pressure measure tube 120 by way of the flow path of the pump 144.

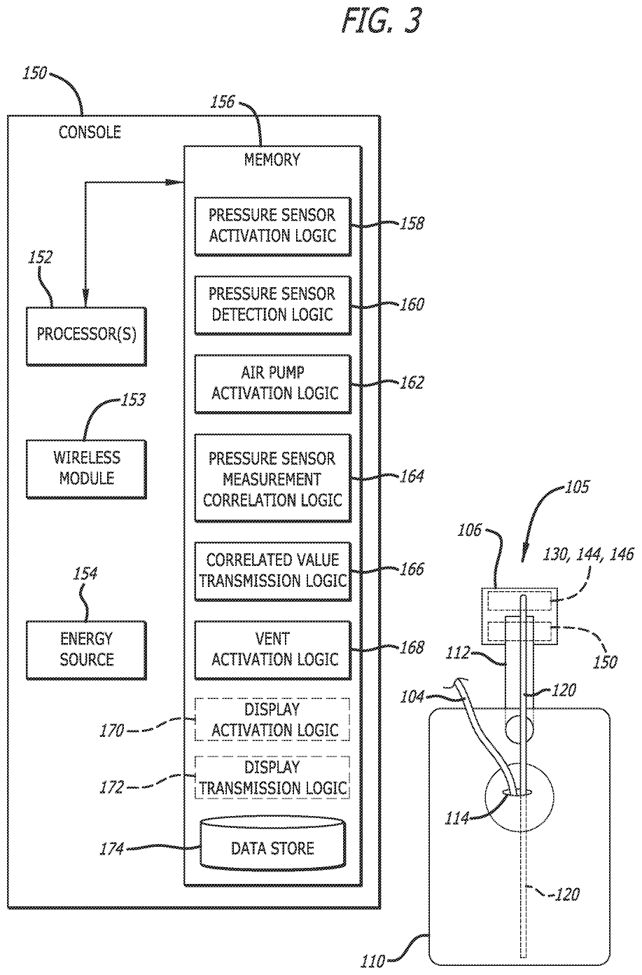

FIG. 3 illustrates a block diagram of some components of the urinary output measuring system 100 including the console 150, in accordance with some embodiments. The console 150 may be in communication with the pressure sensor 130, the valve 146, and the air pump 144. The console 150 may be communicatively coupled with the valve 146, the pressure sensor 130, and the air pump 144 via a wireless or a wired connection. In some embodiments, the console 150 may include a wireless module 153 to facilitate wireless communication with an external computing device or an electronic medical record (“EMR”) system. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like. The console 150 may include one or more processors 152, an energy source 154, a non-transitory computer readable medium (“memory”) 156, and logic stored in the memory 156. In some embodiments, the energy source 154 may be wired to and provide power to each of the pressure sensor 130, the air pump 144, and the valve 146. For description purposes, the logic as described herein is divided into a plurality of logic modules, such as a pressure sensor activation logic 158, a pressure sensor detection logic 160, an air pump activation logic 162, a pressure sensor measurement correlation logic 164, a correlated volume of fluid value transmission logic 166, a valve activation logic 168, a display activation logic 170, a display transmission logic 172, and a data store 174. In some embodiments, the pressure sensor activation logic 158 may be configured to activate the pressure sensor 130. In some embodiments, the pressure sensor activation logic 158 may be activated by a user or by one of the components of the manifold 140 (e.g., the air pump 144) or the computing device. In some embodiments, the pressure sensor activation logic 158 may be activated in accordance with a user-defined time interval (e.g., once every hour, every 5 minutes, or the like).

In some embodiments, the pressure sensor detection logic 160 may be configured to obtain a pressure measurement from the pressure sensor 130 thereby determining the pressure within the pressure measurement tube 120. In some embodiments, the pressure sensor detection logic 160 may be configured to obtain an analog voltage signal from the pressure sensor 130, convert the analog voltage signal into a digital voltage signal and further convert the digital voltage signal into a measured pressure value.

In some embodiments, the pressure sensor detection logic 160 may obtain multiple analog voltage signals, convert the multiple analog voltage signals into multiple digital voltage signals, and further convert the multiple digital voltage signals into measured pressure values. The pressure sensor detection logic 160 may then calculate an average of the multiple measured pressure values to obtain a more accurate measurement of the pressure within the pressure measurement tube 120. In some embodiments, the pressure sensor detection logic 160 may be configured to obtain/record a pressure measurement when the pressure within the pressure measurement tube 120 exceeds a predefined or user-defined pressure value threshold stored in memory 156.

In some embodiments, the air pump activation logic 162 may be configured to activate the air pump 144. The air pump activation logic 162 may also be configured to maintain activation of the air pump 144 for a duration of time. In some embodiments, the air pump activation logic 162 may activate the air pump 144 upon input by the user or in accordance with user-defined time interval (e.g., once an hour, every 5 minutes, or the like). In some embodiments, the air pump activation logic 162 may activate the pressure sensor activation logic 158.

In some embodiments, the pressure sensor measurement correlation logic 164 may be configured to correlate the pressure measurement with a height/depth of the urine with the fluid collection bag 110 as the pressure measurement is related to the hydrostatic pressure of the urine within the fluid collection bag 110. The pressure sensor measurement correlation logic 164 may also determine/calculate the volume of urine within the fluid collection bag 110 in accordance with a geometry of the fluid collection bag 110, where the geometric dimensions may be stored in the memory 156. By way of summary, the pressure sensor measurement correlation logic 164 may determine the volume of urine (i.e., obtain a volumetric measurement of the urine) within the fluid collection bag 110 based on the pressure measurement of the pressure sensor detection logic 160. In some embodiments, the pressure sensor measurement correlation logic 164 may correlate the volumetric measurement with a time of day value, i.e., define a correlated volume of fluid measurement value.

In some embodiments, the correlated volume of fluid value transmission logic 166 may be configured to wirelessly transmit the correlated volume of fluid measurement value and time of day value to the computing device or an electronic medical record (“EMR”) system. In an embodiment, the correlated volume of fluid value transmission logic 166 may be configured to transmit the correlated volume of fluid measurement value and time of day value to a monitor or a display.

In some embodiments, the valve activation logic 168 may be configured to activate the valve 146 to release pressure or air from the fluid measurement tube 120 and/or the fluid collection bag 110 after a measurement has been taken. In some instances, the air pump 144 may over inflate the fluid collection bag 110 resulting in inaccurate volumetric determinations. As such, the valve activation logic 168 may be configured to activate (open) the valve 146 to release the pressure from the fluid collection bag 110. In some embodiments, the vent 116 may prevent over inflation of the fluid collection bag 110.

In some embodiments, the data store 174 may be configured to store/record operational events of the system 100, such as analog voltage measurement values, the digital measurement values, correlated volume of fluid measurement values, and time of day values in memory 156.

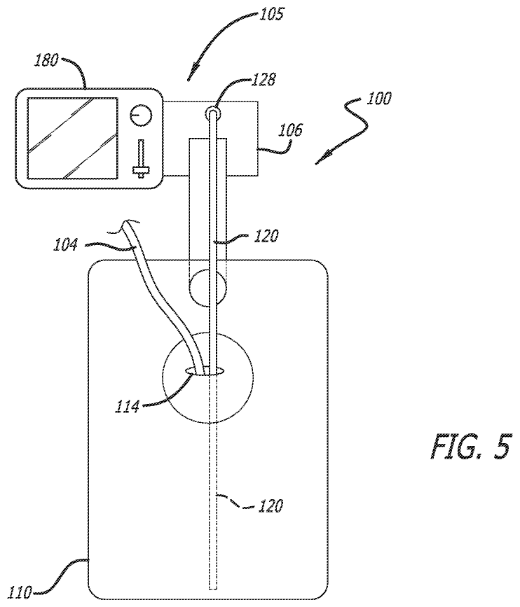

In some embodiments, the console 150 may be in communication with a display 180 (see FIG. 5) configured to display the volume of fluid value corresponding to the volume of fluid within the fluid collection bag 110. In an embodiment, the display 180 may include a touch screen display configured to allow a user to activate the system 100 or define time intervals for detecting the volume of fluid within the fluid collection bag 110. In some embodiments, the display activation logic 170 may be configured to activate the display 180. In some embodiments, the display 180 may continuously display the last volume of fluid value. In some embodiments, the display activation logic 170 may activate the display 180 when the user or the logic detects a new measurement. In some embodiments, the display transmission logic 172 may be configured to transmit the volume of fluid value and the time of day value from the console 150 to the display 180. In some embodiments, a user take a volume of fluid measurement as desired or may configure the system 100 to take a volume of fluid measurement on a user-defined or automated time interval. For example, the system 100 may be configured to take a volume of fluid measurement at two equally spaced time intervals per hour, or a clinician may take a volume of fluid measurement every time the clinician enters into a patient's room. In some embodiments, if the volume of fluid value is above a predetermined or user defined threshold, the console 150 may be configured to transmit an alert (e.g., a text alert including the detected volume of fluid value) to the computing device or EMR system. For example, the clinician may take a size of the fluid collection bag 110 into account and set the user defined threshold to be close to the maximum volume of the fluid collection bag 110.

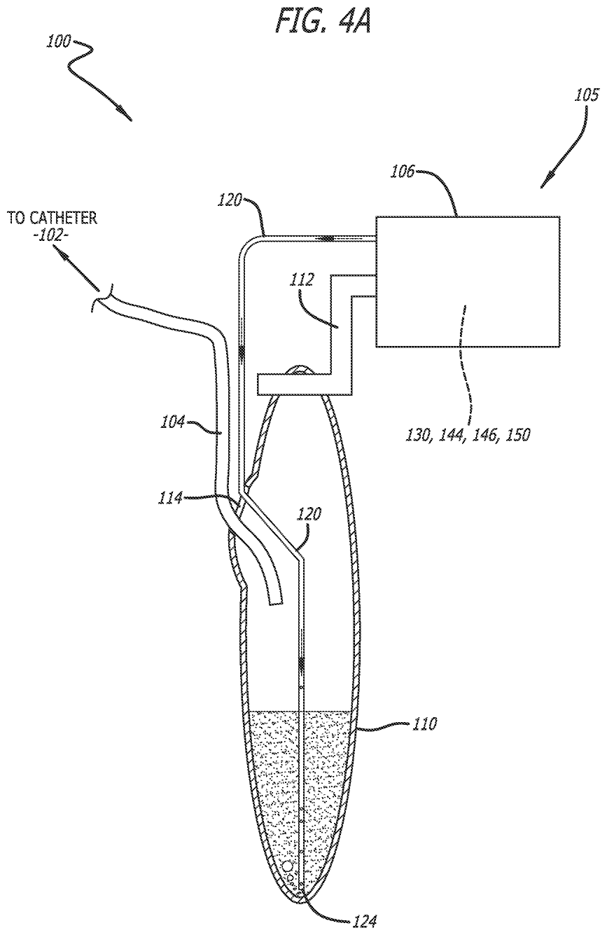

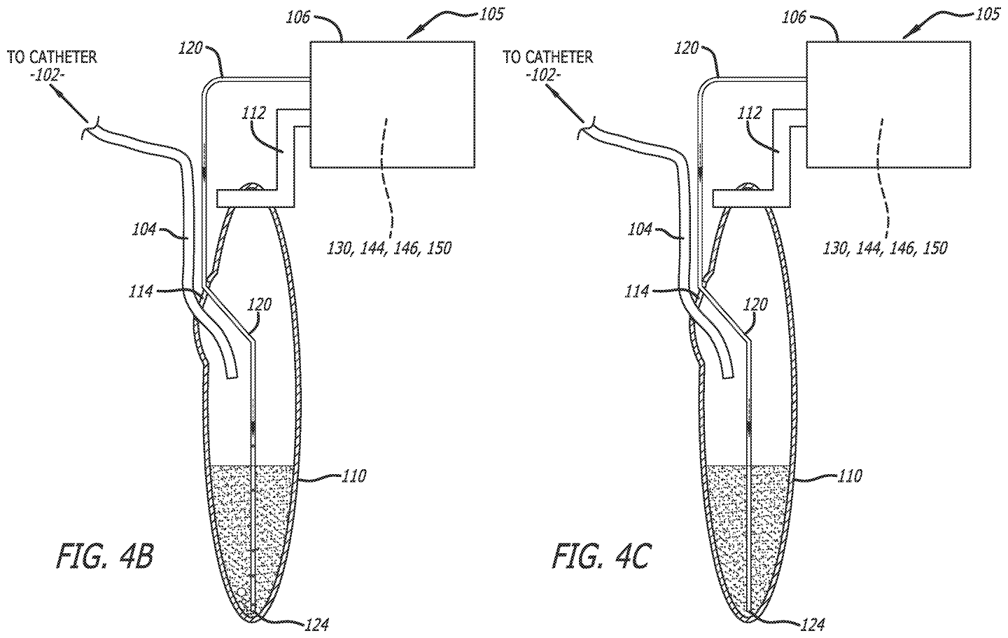

FIG. 4A illustrates an exemplary method of measuring a volume of fluid within the fluid collection bag 110, in accordance with some embodiments. In some embodiments wherein the fluid collection bag 110 is suspended from the fluid collection bag holder 112 and has received therein a volume of fluid from the drainage tube 104. The logic may activate the air pump 144 so that the air pump 144 constantly pumps air into the pressure measurement tube 120 over a defined time interval to purge urine from the pressure measurement tube 120. Some of the air may exit the bottom opening 124 of the pressure measurement tube 120 as bubbles in the urine. The pressure sensor 130 may detect the air pressure within the pressure measurement tube 120 over the time interval and communicate the detected pressure values to the logic. In some embodiments, activating when the air pump 144 may activate the pressure sensor 130. The maximum air pressure within the pressure measurement tube 120 during and immediately after purging of the pressure measurement tube 120 may relate to the hydrostatic pressure of the urine within the fluid collection bag 110. As the urine is purged from the pressure measurement tube 120, the pressure within the pressure measurement tube 120 may rise to a maximum value when all urine is purged from the pressure measurement tube 120 at which point the pressure as detected by the pressure sensor 130 is related to the maximum hydrostatic pressure. The maximum hydrostatic pressure may be approximated by averaging the multiple pressure measurements that may include crest and trough pressures of detected pressure oscillations over a period of time. The detected pressure values may be correlated with the volume of urine as described above. The valve 146 may be activated to release air pressure from the pressure measurement tube 120 and/or from the fluid collection bag 110. The system 100 may perform multiple measurements to determine the volume of urine within the fluid collection bag 110 at different times or to determine a rate of urine output over a period of time.

FIGS. 4B-4C illustrate another exemplary method of measuring the volume of fluid within the fluid collection bag 110, in accordance with some embodiments. As illustrated in FIG. 4B when the fluid collection bag 110 has received therein a volume of fluid from the catheter 102, the logic may activate the air pump 144. The air pump 144 may constantly pump the air through the pressure measurement tube 120 over a time interval into the volume of fluid to constantly purge the pressure measurement tube 120 of fluid. As such, some air may be pushed out of the bottom of the pressure measurement tube 120 into the urine as bubbles.

As illustrated in FIG. 4C, the pressure sensor 130 may be configured to detect the air pressure within the pressure measurement tube 120 over the time interval once the fluid has been purged from the pressure measurement tube 120 and communicate the detected pressure values to the logic. The maximum air pressure within the pressure measurement tube 120 immediately after purging may be equal to the hydrostatic pressure of the volume of urine within the fluid collection bag 110. The maximum air pressure may oscillate near the maximum hydrostatic pressure as the bubbles continually form and collapse. Once the air pump 144 is deactivated and bubbling is complete, the pressure values detected within the pressure measurement tube 120 may be consistent (i.e., not oscillate) and may be detected by the pressure sensor 130. The pressure values may be correlated with volume of fluid values as described above. As additional urine is captured within the fluid collection bag 110 causing the hydrostatic pressure to increase, urine may enter the pressure measurement tube 120. When an additional measurement is desired, the air pump 144 may be activated to re-purge urine from the pressure measurement tube 120. With the urine re-purged from the pressure measurement tube 120, the pressure within the pressure measurement tube 120 may be related to the hydrostatic pressure of the increased volume of urine within the fluid collection bag 110. The new detected pressure values will may then be correlated with the increased volume of fluid values to update the determined volume of fluid within the fluid collection bag 110.

FIG. 5 illustrates a plan view of the urinary output measuring system 100 that, in accordance with some embodiments, further includes a display 180. The display 180 is couple with the console 150. In some embodiments, the display may be included with the housing 106 of the system module 105. In other embodiments, the display 180 may be coupled with the housing 106. The display 180 may be configured to display information as defined by the logic, such as the correlated volume of fluid value, an average volume value, the rate of capture of fluid within the fluid collection bag 110, or a warning to dispose of the captured volume of fluid if the captured volume of fluid has reached the maximum capacity of the fluid collection bag 110, for example. In some embodiments, the display 180 may be configured to display the correlated volume of fluid values on the display upon the console 150 upon the detecting and correlating the volume of fluid values.

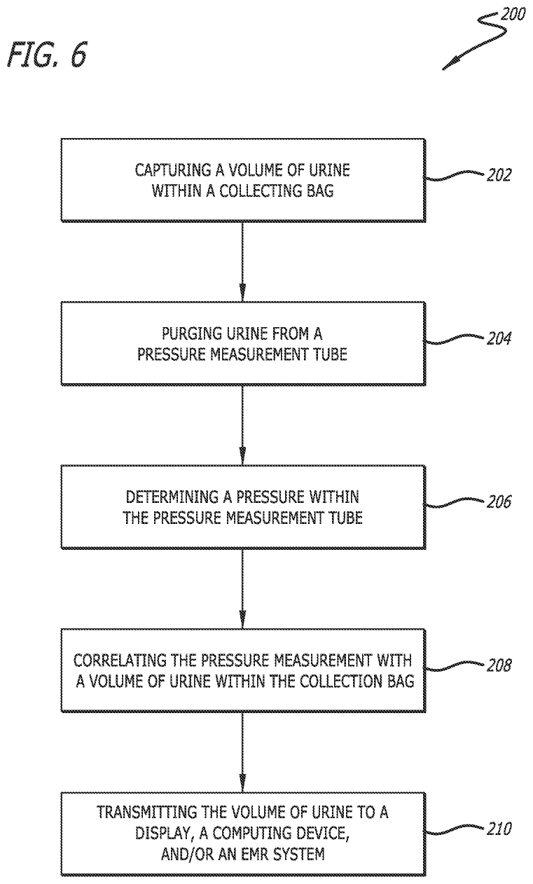

FIG. 6 illustrates a flow chart of the exemplary method 200 of measuring the volume of urine within the fluid collection bag 110 that, in accordance with some embodiments, may include all or a subset of the following steps or processes. In some embodiments, the method 200 includes capturing a volume of urine within the fluid collection bag 110 (block 202). In some embodiments, capturing the volume of fluid within the fluid collection bag 110 includes capturing the volume of fluid within the fluid collection bag 110 by gravity flow. In some embodiments, capturing the volume of fluid within the fluid collection bag 110 includes the receiving urine from urinary catheter coupled with a drainage tube extending to the fluid collection bag 110.

The method 200 further includes purging the pressure measurement tube 120 of urine (block 204). In some embodiments, purging the pressure measurement tube 120 includes activating an air pump 144 to pump air into the pressure measurement tube 120 to purge the urine from the pressure measurement tube 120. In some embodiments, purging the pressure measurement tube 120 includes causing air to exit the pressure measurement tube 120 as bubbles passing through the urine within the fluid collection bag 110.

The method 200 further includes obtaining a pressure measurement from the pressure sensor that is related to a maximum hydrostatic pressure of the urine (block 206). In some embodiments, obtaining a pressure measurement may include obtaining multiple pressure measurements over a time period and calculating an average of the multiple pressure measurements. In some embodiments, obtaining multiple pressure measurements over a time period may occur while the urine is being purged from the pressure measurement tube 120, i.e., while the air pump is activated. In other embodiments, obtaining a pressure measurement may include obtaining the pressure measurement after the urine has been purged from the pressure measurement tube 120, i.e., after the air pump is deactivated.

In some embodiments, the method 200 includes correlating the detected pressure value with a volume of urine collected within the fluid collection bag (block 208). In some embodiments, method includes calculating the volume of the urine based on the pressure measurement and a geometry of the bag.

In some embodiments, the method 200 includes transmitting one or more volume of urine values to the computing device, the display 180, or the EMR system (block 210). In some embodiments, transmitting the one or more volume of fluid values includes transmitting the one or more volume of urine values as the one or more volume of urine values are detected or on a user-defined time interval.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A urinary output measuring system, comprising:
a collection container configured to receive therein urine from a patient via a urinary catheter;
a pressure measurement tube defining a first end and an opposite second end, the second end disposed within the collection container such that the second end is located at the bottom of the collection container; and
a system module comprising:
a pressure sensor in fluid communication with the pressure measurement tube so as to detect a pressure within the pressure measurement tube;
an air pump fluidly coupled with the pressure measurement tube adjacent the first end via an output port; and
a console operatively coupled with the pressure sensor and the air pump, the console including one or more processors and a non-transitory computer-readable medium having stored thereon logic that, when executed by the one or more processors, causes operations of the system that include determining a volume of the urine collected within the collection container, wherein determining a volume of the urine collected within the collection container includes:
activating the air pump to cause a flow of air from the first end to the second end of the pressure measurement tube, thereby purging the urine from the pressure measurement tube;
obtaining a pressure measurement of the air when the urine is purged from the pressure measurement tube, the pressure measurement defined by a hydrostatic pressure of the urine collected within the collection container; and
calculating a volume of the urine collected within the collection container based on the pressure measurement in combination with a geometry of the collection container.

2. The system according to claim 1, further comprising a display coupled with the console, the operations further including indicating the volume of the urine on the display.

3. The system according to claim 1, wherein the console further includes a wireless module, the operations further including wirelessly communicating the volume of the urine to at least one of a computing device or an electronic medical record system.

4. The system according to claim 1, wherein:
the system module further includes a housing for containing the pressure sensor, the air pump, the console, and the display, and
the housing is configured to attach to a bed.

5. The system according to claim 4, wherein the collection container is configured to attach to the housing such that the collection container is suspended from the housing.

6. The system according to claim 5, wherein the container includes a hook and the housing includes a hook pocket configured to receive the hook.

7. The system according to claim 1, wherein:
the collection container defines a closed volume, and
the pressure measurement tube extends through a wall of the collection container such that the pressure measurement tube forms a seal with the wall.

8. The system according to claim 1, further including a drainage tube fluidly coupleable with the collection container, the drainage tube coupleable with the urinary catheter to enable flow of the urine from the catheter to the collection container.

9. The system according to claim 8, wherein the drainage tube is fixedly attached to the collection container.

10. The system according to claim 1, wherein:
the collection container includes a vent configured to enable passage of air out of the collection container,
the vent includes a vent filter extending across a flow path of the vent,
the vent filter defines a sterile barrier, and
the vent filter includes a hydrophobic membrane.

11. The system according to claim 1, wherein the air pump includes a pump filter extending across a flow path of the air pump, the pump filter defining a sterile barrier.

12. The system according to claim 1, wherein the collection container is a flexible bag.

13. The system according to claim 1, further including valve fluidly coupled with the pressure measurement tube such that, when the valve is in an opened state, air is allowed to flow into and out of the pressure measurement tube, wherein:
the valve is operatively coupled with the console,
the logic is configured to transition the valve between the opened state and a closed state, and
the operations further include:
transitioning the valve from the opened state to the closed state before activating the air pump; and
transitioning the valve from the closed state to the open state after obtaining a pressure measurement.

14. The system according to claim 13, wherein the valve includes a valve filter extending across a flow path of the valve, the valve filter defining a sterile barrier.

15. The system according to claim 1, wherein:
obtaining a pressure measurement of the air includes:
obtaining a plurality of pressure measurements, and
calculating an average of the plurality of pressure measurements, and
calculating a volume of the urine collected within the collection container includes calculating a volume of the urine based on the average of the plurality of pressure measurements.

16. The system according to claim 13, further comprising a manifold having a frame structure and a number of fluid passageways that define the fluid communication between pressure measurement tube and each of the pressure sensor, the air pump, and the valve, wherein the pressure sensor, the air pump, and the valve are fixedly attached to the manifold, and wherein the pressure measurement tube is detachably coupled with the manifold.

17. The system according to claim 1, wherein the operations further include determining the volume of the urine collected within the collection container multiple times according to a predefined determination schedule.

18. The system according to claim 1, wherein the operations further include determining the volume of the urine collected within the collection container in response to an input from the user.

19. A method of determining a volume of urine collected within a bag, comprising:
purging urine from a pressure measurement tube extending between a pressure sensor and a bottom interior of the bag;
obtaining a pressure measurement from the pressure sensor, the pressure measurement related to a hydrostatic pressure of the urine; and
calculating the volume of the urine based on the pressure measurement and a geometry of the bag.

20. The method according to claim 19, wherein purging the urine from the measurement tube includes pumping sterile air into the tube so that air bubbles exit the tube at the bottom interior of the bag.
